(19) United States
(12) Patent Application Publication
(10) Pub. No.: US 2026/0083884 A1
(43) Pub. Date: Mar. 26, 2026

(54) ULTRAHYDROPHOBIC COATINGS ON DEVICE CUTTING ELEMENTS

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Teo Heng Jimmy Yang, Heath (GB)

(21) Appl. No.: 19/409,596

(22) Filed: Dec. 4, 2025

Related U.S. Application Data
(63) Continuation of application No. 17/656,022, filed on Mar. 23, 2022.
(60) Provisional application No. 63/175,934, filed on Apr. 16, 2021.
(60) Provisional application No. 63/201,322, filed on Apr. 23, 2021.

(51) Int. Cl. A61L 31/10; A61B 17/29; A61B 18/14

(52) U.S. Cl. CPC A61L 31/10; A61B 17/29; A61B 18/1445

(57) ABSTRACT

Various embodiments disclosed relate to a non-stick layer for cutting elements on electrosurgical cutting tools. The present disclosure includes systems, devices, and methods of making and using a non-stick layer on cutting elements. The non-stick layer can include coatings, surface structures, or combinations thereof. In one example, the medical device includes a hydrophobic coating with a hydrophobic nanoscale physical structure. In one example, the medical device includes a heated cutting assembly, such as a resistive heated cutting assembly.

### CROSS-REFERENCE TO RELATED APPLICATIONS

This application is a Continuation of U.S. patent application Ser. No. 17/656,022, filed Mar. 23, 2022, which claims the benefit of priority to U.S. Provisional Patent Application Ser. No. 63/175,934, filed Apr. 16, 2021 and U.S. Provisional Patent Application Ser. No. 63/201,322, filed Apr. 23, 2021, the contents of which are incorporated herein by reference in their entireties.

### TECHNICAL FIELD

This document pertains generally, but not by way of limitation, to electrosurgical devices that can be used for various surgical procedures.

### BACKGROUND

Electrosurgery uses the application of a high frequency alternating polarity electrical current in combination with mechanical cutting elements such as blades to cut, coagulate, desiccate, or fulgurate tissue. The high frequency alternating current (AC) can be converted to heat by resistance as it passes through tissue. The result of heat buildup within the tissue can be used to cause tissue thermal damage, resulting in effects such as cutting or cautery of tissue. Electrosurgery can allow for high precision cutting in surgery with low blood loss. In some cases, mechanical cutting elements can be used in an alternating fashion or assisted with radio frequency (RF) energy.

Several medical devices will benefit from a reduction in adhesion of material to one or more surfaces. For example, in medical cutting devices coagulation and/or tissue may adhere to a cutting assembly and reduce the efficacy of a cutting operation. In particular, heated cutting assemblies, such as resistive heated blades will benefit from reduced adhesion. Improved medical cutting devices and other medical devices with reduced adhesion surfaces are desired.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic diagram of an electrosurgical system including a surgical device with a cutting element in accordance with some example embodiments.

FIG. 2 illustrates a distal portion of a surgical jaw with a non-stick layer in accordance with some example embodiments.

FIG. 3 illustrates a distal portion of a dissecting forceps with a non-stick layer in accordance with some example embodiments.

FIG. 4 illustrates a distal portion of a surgical pencil with a non-stick layer in accordance with some example embodiments.

FIG. 5 illustrates a distal portion of a laparoscopic loop with a non-stick layer in accordance with some example embodiments.

FIG. 6 shows a medical cutting device in accordance with some example embodiments.

FIG. 7 shows a cutting assembly of the medical cutting device from FIG. 6 in accordance with some example embodiments.

FIG. 8 shows a cross section of a cutting assembly in accordance with some example embodiments.

FIG. 9 shows a cross section of another cutting assembly in accordance with some example embodiments.

FIG. 10 is a schematic diagram of a surface on a surgical device coating with a hydrophobic coating in accordance with some example embodiments.

FIG. 11 shows another surface including a hydrophobic physical structure in accordance with some example embodiments.

FIG. 12 is a flow chart depicting a method of applying a hydrophobic coating to a surgical device in accordance with some example embodiments.

FIG. 13 shows a flow diagram of an example method in accordance with some example embodiments.

### DETAILED DESCRIPTION

The present disclosure provides methods for coating a cutting component of a surgical device, and a coated cutting element of a surgical device. The surgical device can be, for example, a forceps with a blade, a dissection forceps, a laparoscopic loop, a spatula, or other cutting electrosurgical instrument. In such cutting devices, such as blood vessel sealing devices, tissue build-up can occur on the cutting component, such as blades. A build-up of tissue on mechanical cutting components can cause tissue sticking and tearing during operation and reduce the effectiveness of cutting techniques. This can occur when the tissue exhibits latent heat from vessel sealing plates.

Discussed herein, the use of hydrophobic layers on such mechanical cutting elements can help reduce tissue build-up and tissue sticking. In some cases, the mechanical cutting elements can be radio frequency (RF) assisted mechanical cutting elements. The hydrophobic layers can include coatings, surface structures, or combinations thereof, that serve as a non-stick layer on the cutting element. For example, coatings with polysiloxanes or fluorosilanes can be used. Optionally, such hydrophobic layers can be used to lubricate the channel in which the cutting element resides.

For example, where a forceps vessel sealing device is used, tissue and blood build up can occur on and around the forceps jaw. Additionally, the mechanical cutting element itself, such as a blade between the jaw, is susceptible to tissue build up. This can affect the performance of the cutting edge of the blade. With a vessel sealing forceps, as the vessel sealing plates are used, they can heat the surrounding area, which can include where the cutting blade is operating. In some cases, tissue that has just been sealed can be warm, with denatured collagen and proteins. As the blade passes through such tissue to make a cut, the denatured collagen and proteins can stick to the cutting blade. Sticking can happen even with tissue that hasn't been sealed or otherwise heated but is still being cut. Deposits on the cutting blade can increase the required cut trigger force to advance the blade through the tissue, can affect cut quality, and can-do damage to the seal itself.

Application of a non-stick layer, such as a coating or surface structure, can reduce this tissue sticking. This can help preserve the quality of trigger actuation forces and cut quality. The non-stick layer can be, for example, a nano-coating of polysiloxanes or fluorosilanes, or an etched surface providing nanostructures. In some cases, the channel in which the cutting element resides can also be coated.

In an example, an electrosurgical device can include a longitudinal shaft having a distal portion and a proximal portion, the shaft for at least partial insertion into a patient, an end effector on the distal portion of the longitudinal shaft, wherein the end effector is configured to cut tissue, the end effector having an electrically conductive component configured to operably couple to a source of electrosurgical energy for treating tissue, and a cutting element and a non-stick layer at least partially covering the cutting element, wherein the non-stick layer comprises a material having a surface adherence to tissue that is less than a surface adherence to tissue of the material of the cutting element.

In an example, a method can include applying a non-stick layer to a mechanical cutting element of an end effector in a surgical device, wherein the non-stick layer comprises a hydrophobic surface.

Discussed herein are methods for coating a mechanical cutting component of a surgical device, and a coated cutting portion of a surgical device. The surgical device can be, for example, a vessel sealing device, including a mechanical cutting portion between the jaws. In some cases, cutting portion (such as a blade) can be subject to sticking against tissue being operated on. This can cause tissue tearing during operation. For this reason, proposed herein is the use of a non-stick, hydrophobic surface on the mechanical cutting portion. This may be provided by, for example, physically modifying the external surface of a mechanical cutting portion by laser texturing or etching the surface into a hydrophobic microstructure or nanostructure. In another example, the hydrophobic surface may be provided by applying a hydrophobic coating on the mechanical cutting portion, such as, for example, a thin coating made of a material such as polysiloxanes or fluorosilanes.

Mechanical cutting portions of electrosurgical devices, and associated channels, can be subject to build-up of tissue during surgical procedures. For example, tissue heated during vessel sealing or other coagulation, can stick to mechanical cutting elements such as blades, forceps, loops, or pencils, in addition to blade channels and mechanisms.

For example, as vessel sealing devices are exposed to blood and other bodily tissues, they are apt to build-up of these materials in and around the jaws of the vessel sealing device. Similarly, the cutting element, such as the blade, is also subject to build-up, such as when the blade is subject to increased heating and prone to sticking due to transferred thermal energy. A vessel sealing device can be used, for example, to apply radio frequency (RF) energy with electrodes to the target tissue, leaving two seals. Subsequently, a mechanical blade can be used to cut and separate the seals. Tissues that have been acted on, and tissues nearby the cutting blade that have not been acted on, can potentially stick to the blade as it moves in and out for the cut. As this tissue builds up and adheres to the blade, greater force is required to advance the blade through the tissue. This can also deteriorate cut quality, resulting in stretched and ragged cut edges instead of sharp edges.

Applying a hydrophobic layer, such as a coating or surface structure, to the blade can prevent sticking and reduce incidences of increased cut trigger actuation forces. Similarly, the pathways through which the blade moves can be coated, allowing lubrication and prevent tissue build-up in the channel impeding the mechanical cut blade. In some cases, an etched surface pattern can be used to achieve the non-stick effect, instead of a coating, or in combination with a coating. Such a hydrophobic layer can also help reduce undesired cutting element heating. These types of hydrophobic layers can be applied to mechanical cutting elements such as blade, dissection forceps, and stationary active bipolar cut electrodes.

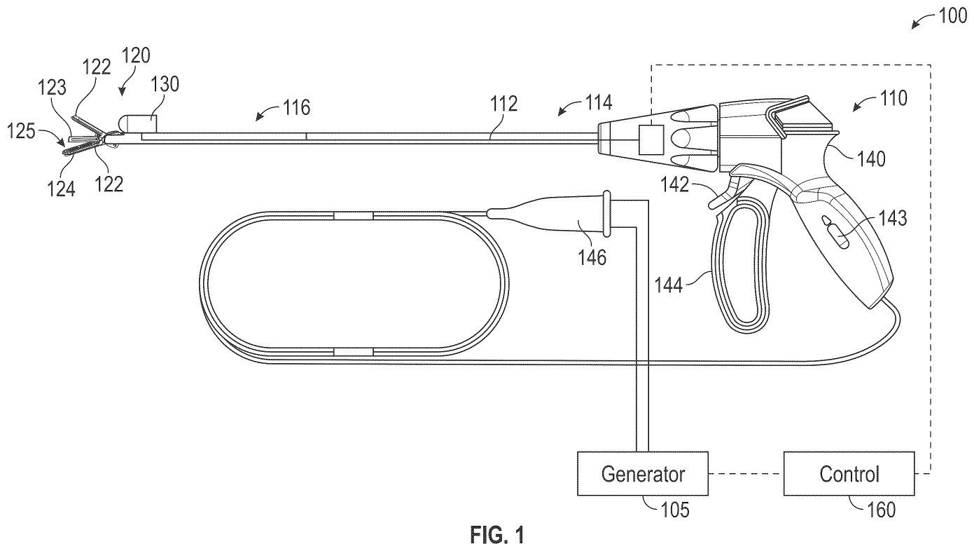

FIG. 1 illustrates a schematic diagram of an example of portions of an electrosurgery system 100, such as can include an electrosurgery device 110 with an electrosurgical end effector 120 such as forceps, spatula, loop or another cutting device, such as containing a blade. The device 110 can be connected to an electrosurgical energy generator 105 and a controller 160.

The electrosurgery device 110 can include a longitudinal shaft 112 having a proximal portion 114 and a distal portion 116. The distal portion 116 can include an end effector 120, such as which can include an insulation element 123, electrodes 124, and a non-stick layer 125. A proximal portion 114 of the device can be connected to a handpiece 140, such as with actuators 142, 143, and 144. The device 110 can also include a connector 146 such as can be configured to be connected to the generator 105.

The generator 105 can be external to but coupled to the electrosurgical device 110. The generator 105 can provide electrical energy to the end effector 120 of the electrosurgical device 110, such as through the electrical connector 146. The electrical generator 105 can produce a current deliverable by the end effector 120 such as for inducing a coagulation mode of electrosurgery. The electrical generator 105 can be in communication with the controller 160, which can direct the application of electrosurgical energy to the end effector 120 in the electrosurgical device 110.

The type and amount of electrical energy provided by the generator 105 can vary, such as depending on the desired treatment. The electrosurgical waveform produced, the voltage, and the power of the electrosurgical energy being delivered, and the size and surface area of the end effector 120, can affect the depth and the rate of producing heat, which, in turn, can alter the final effect on the target tissue.

The electrosurgery device 110 can include a bipolar or monopolar electrosurgery end effector 120 such as for applying high-frequency alternating polarity electrical current to biological tissue, such as to cut, coagulate, desiccate, or fulgurate the tissue, as may be desired by the surgeon treating the patient.

The electrosurgery device 110 can include a wet field device such as for wet field electrosurgery, such as in a saline solution, or in an open wound. In a wet field device, heating can result from an AC current passing between two electrodes. Heating can be the greatest where the current density is the highest. Thus, smaller surface area electrode can produce a greater amount of heat for treating tissue.

In the device 110, the shaft 112 with the proximal portion 114 and the distal portion 116 can be sized, shaped, or arranged for partial insertion of the device 110 into a patient. The shaft 112 can include or can be made of one or more of a composite, plastic, or metallic material, or other material suitable for surgical applications. The proximal portion 114 can be near an operator, such as a surgeon, when the device 110 is in use. In some cases, the operator can be a robotic arm or other machine. The distal portion 116 can be sized, shaped, or arranged for insertion into the patient so that distal portion 116 is further from the operator during use.

In some cases, the shaft 112 can be sized, shaped, arranged, or otherwise configured for laparoscopy, in some cases, the shaft 112 can be shorter such as for open surgery applications. In some cases, such as for laparoscopy, the shaft can be long. In an open surgery application, the shaft can include a tissue interface element with cutting, coagulating, and sensing elements in or on a distal portion of that device.

Laparoscopy can include, for example, a surgical procedure in which a small incision is made, through which a device is inserted to diagnose or treat conditions. Laparoscopy is considered less invasive than regular open abdominal surgery. In the case of laparoscopy, an optical visualization or imaging device may also be inserted along with the device 110, such as to permit the optical device to allow viewing or imaging such as for the operator to observe the tissue. The optical visualization or imaging device can include a laparoscope, or viewing tube, such as with a camera. In some cases, the optical visualization or imaging device can include an ultrasound type imaging device for the operator to use during treatment.

By contrast, open surgery approaches can involve a larger incision, such as can allow more direct visual observation of cutting of skin and tissue, such to permit the surgeon to have a fuller view of the structures and organs involved in the procedure.

For example, in some applications, the shaft 112 can have a length in a range of 10 mm to 30 mm, inclusive. The shaft 112 can be narrow in a cross-section or a lateral dimension, such as for patient insertion via an incision. For example, the shaft 112 can have a cross-sectional or lateral width in a range of less than 6 mm, inclusive.

The end effector 120 can be located at or near the distal portion 116 of the shaft 112. The end effector 120 can include a bipolar or monopolar electrode and optionally a blade, such as for use in cutting tissue. Bipolar or monopolar electrodes can make use of high frequency electrical current such as to cut, coagulate, desiccate, or fulgurate tissue. With a bipolar electrode configuration, current passes through the tissue between two more closely-spaced electrodes, such as between individual electrode arms of a forceps-type electrode. In a bipolar configuration, the current passes through the tissue between tips of two active electrodes, such as between electrode tips of a bipolar forceps. With a monopolar configuration, current can pass through the tissue between the end effector 120 and a pad on the patient's abdomen or other, separate return electrode. The electrical generator 105 can be connected to both active and return electrodes, such as for sending and receiving current. The end effector 120 can be configured to heat the targeted tissue.

Tissue can see a reduction in resistance as it heats, as the fluid content remains unaltered and does not change to the more resistive state of steam. Thus, during a procedure the risk increases of moving into the undesirable lower resistance range during the heating of some tissues. By increasing the resistance of the devices by about 5 to 10 Ohms, it can have a large impact on a device when connected to a limited source current generator, by essentially moving the power curve towards the lower resistance states.

The handpiece 140 can include one or more user-actuators, such as the actuators, 143, 144. In some cases, these can include one or more of levers, buttons, wheels, switches, triggers, or a combination thereof. One of the actuators 142, 143, 144, can provide a user-interface to control a first switch that selectively connects the end effector 120 to the generator 105 or other circuitry that can provide electrosurgical energy to the first end effector 120 such as for cutting and coagulation. Additional actuators, such as buttons, triggers, or other user-actuatable mechanisms can be included on the handpiece 140 of the device 110 or elsewhere for surgeon use, such as for direction and action of the end effector 120, movement of the shaft 112, or one or more other operations of the device 110.

The electrosurgical device 110, including the triggers on the handpiece 140, the end effector 120, and the one or more sensors 130, can be in communication with the controller 160. The generator 105 can also be in communication with the controller 160.

The controller 160 can include a processor and a memory such as to permit the controller 160 to communicate with and control the generator 105. The controller 160 can be used to allow for both predictive and reactive control of the duty cycle produced by the generator 105.

The controller 160 can operate as a standalone device, or may be networked to other machines. The controller 160 can include a hardware processor, such as a central processing unit (CPU), a graphics processing unit (GPU), a hardware processor core, or any combinations thereof. The controller 160 can further include a memory, including a main memory and a static memory. The controller 160 can include an input device, such as a keyboard, a user interface, and a navigation device such as a mouse or touchscreen.

The controller 160 can additionally include a storage device, a signal generation device, a network interface device, and one or more sensors. The storage device can include a machine readable medium on which is stored one or more sets of data structure or instructions embodying or utilized by any one or more of the techniques described herein. The instructions may also reside, completely or at least partially, within the main memory, within static memory, or within the hardware processor during execution thereof by the controller.

In an example, one or any combination of the hardware processor, the main memory, the static memory, or the storage device may constitute machine readable media, that may include any medium that is capable of storing, encoding, or carrying instructions for execution by the controller 160 and that cause the controller 160 to perform any one or more of the techniques of the present disclosure, or that is capable of storing, encoding or carrying data structures used by or associated with such instructions. Non-limiting machine readable medium examples may include solid-state memories, and optical and magnetic media. The instructions on the controller 160 may further be transmitted or received over a communications network using a transmission medium via a network interface device.

The device 100 in FIG. 1 can be used, for example, to cut tissue, such as a mechanical cutting device assisted with RF (radio frequency) energy derived from the current produced by the generator. For cutting tissue, the generator 105 can produce an electrosurgical energy waveform similar to a sine wave. Cutting can use a continuous electrosurgical energy waveform such as can be able to apply the maximum output power of the generator 105, if desired. By comparison, for coagulating tissue, the peak current output can be greater than for cutting tissue, but with an intermittent or lower duty cycle waveform with lower average power than a cutting waveform. For cutting tissue, the peak voltage can be greater.

Cutting tissue can include resection and dissection. Various electrosurgical waveforms can be used for electrosurgical procedures. Rapid heating of tissue using a continuous waveform can result in vaporization, fragmentation, and ejection of tissue fragments, allowing for tissue cutting. Open circuit voltage of such electrical waveforms can be, for example, from about 300 to about 10,000 V peak-to-peak, inclusive. In some cases, rapid tissue heating can allow for explosive vaporization of interstitial fluid; if the voltage is sufficiently high, such as above 400 V peak-to-peak, the vapor can be ionized, sometimes resulting in conductive plasma allowing flow of electric current from the electrode via the plasma into the tissue.

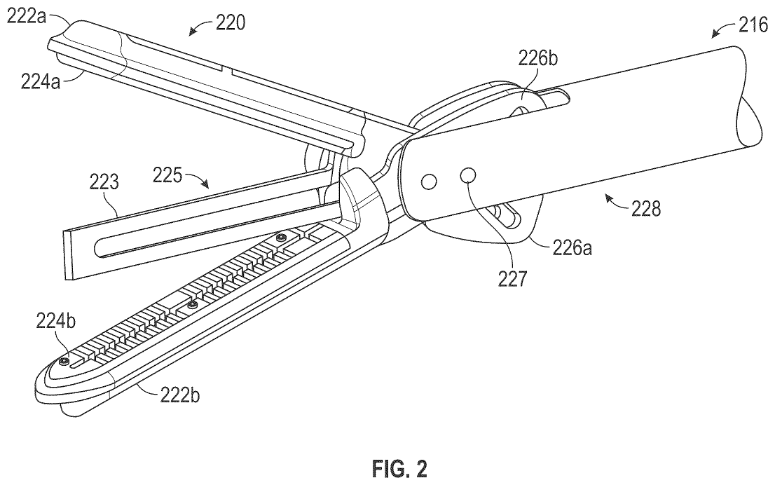

Shown in FIG. 1, the end effector 120 can be a surgical cutting device, such as surgical forceps with a central blade, or other RF type cutting device, such as the examples discussed below with reference to FIGS. 2-5. In other cases, the device can be a spatula, loop, dissecting forceps, or other cutting instrument having a cutting element 123 on or near the cutting end of the device.

During operation, build-up of tissue on the cutting element can sometimes cause a reduced quality of cut. For example, in system 100, a vessel sealing forceps is shown as the end effector 120. The sealing forceps can include a jaw 121 with a cutting element 123 therebetween. During operation, tissue may be sealed by the forceps and consequently heated. Subsequently, the tissue can be mechanically cut with the cutting element 123. In this case, heated tissue can stick to the cutting element 123. Such sticking can also occur on these types of mechanical cutting elements 123 that are not subject to energy application during sealing and consequent heating. In any case, tissue, blood, proteins and/or other such materials can cover the cutting edges of the cutting element 123 and reduce the precisions of the cut. Additionally, this can sometimes require additional force in application of the cutting element 123 to get through the tissue. Moreover, stuck tissue can sometimes tear when sticking to the cutting element 123.

Thus, the end effector 120 can further include a non-stick layer 125. The non-stick layer 125 can at least partially cover the cutting element 123. The non-stick layer 125 can have a surface adherence less than that of the cutting element 123. In some cases, the non-stick layer 125 can be a coating, such as polydimethylsiloxane, hexadimethylsiloxane, or tetramethyldisiloxane. In an example, the non-stick layer 125 can have a thickness in range of about 10 nm to about 30 nm. In some cases, the non-stick layer 125 can have a substantially uniform thickness. In some cases, the non-stick layer 125 can have a non-uniform thickness. In some cases, the non-stick layer 125 can be discontinuous. In some cases, the non-stick layer can be continuous. The non-stick layer 125 can include one or more asperities, such as nanoparticles. The non-stick layer 125 can include an electrically insulated or a non-conductive material. In some cases, the non-stick layer 125 can include a hydrophobic surface structure, a coating, or a combination thereof. In some cases, the non-stick layer 125 can overlap a portion of the electrode 124.

This layer 125 can be chosen and applied to the cutting element 123 of the end effector 120 to prevent or reduce sticking of tissue to the cutting element 123 of the device 110. The non-stick layer 125 can, for example, include a nanostructure and a non-stick structure to reduce sticking of the coagulum to the tip of the device 110. The non-stick layer 125 can, for example, have a low surface energy, such as to prevent sticking of coagulum to the device. The non-stick layer 125 can allow for reduction of thermal transfer between the cutting element 123 and the target tissue, so as to reduce sticking therebetween. In some cases, the non-stick layer 125 can include super hydrophobic materials.

An example forceps end effector 120 is shown in greater detail in FIG. 2. The end effector 120 can extend distally from the shaft. The end effector 120 can include a cutting element 123 and electrodes 124, along with layer 125. The end effector 120 can be used, for example, for surgery such as colon surgery or intestinal surgery. The end effector 120 can be used for cutting tissue. The end effector 120 can further include the non-stick layer 125.

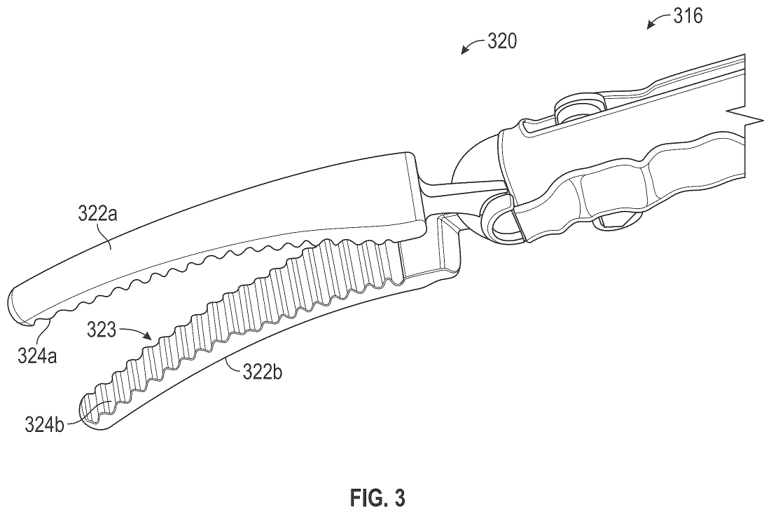

The cutting element 123 can be, for example, a cutting edge, blade, spatula, or other mechanical cutting component. The cutting element 123 can be configured for use in conjunction with one or more electrodes, such as electrode plates on a forceps. The cutting element 123, however, can be used for mechanical separation of target tissue, as opposed to treatment with electrical current to cut or coagulate tissue. At varying points during the operation, the surgeon may desire to mechanically separate tissue using the cutting element 123. Various types of electrosurgery devices with differing cutting elements are discussed below with reference to FIGS. 3-5.

In an example, the layer 125 can include a non-electrically conductive coating on an external surface of the end effector 120, or an insulative coating. The layer 125 can have a resistance or impedance of less than about 10 ohms, or less than about 5 ohms. The layer 125 can have a surface adherence less than that of the electrode material 124. The layer 125 can, for example, have a coefficient of friction that is lower than that of the cutting element 125.

In some cases, the coating can be uniform in coverage and thickness, in some cases it can be fully or partially coating the end effector 120. The layer 125 can have a thickness of up to about 300 nm, up to about 200 nm, up to about 100 nm, or less. In some cases, the layer 125 can be hydrophobic or superhydrophobic. In some cases, the layer 125 can have a nanostructure or microstructure to reduce stickiness.

Examples of the present disclosure provide for disposing a non-stick coating on components of an electrosurgical device at a particular thickness or within a particular range of thicknesses such that the non-stick coating provides adequate tissue sticking reduction during tissue sealing without negatively impacting tissue sealing performance of the device.

As discussed herein, a thickness of the non-stick coating can be in the range of 10 nm to about 300 nm and provide non-stick benefits. However, while non-stick properties can be provided, various portions of this range can provide additional benefits, while still providing tissue adhesion resistance and sensing capability. In one example, the non-stick coating can be a thin coating, e.g., having a thickness in the range of, but not limited to, about 10 nm to about 30 nm. In one example, the non-stick coating has a thickness in the range of about 10 nm to about 20 nm. In one example, the non-stick coating has a thickness less than 20 nm such as about 15 nm.

The non-stick layer 125 can allow passage of energy, such as RF energy, through such that the end effector 120 can affect the target tissue. For example, the layer 125 can be a light capacitive element or light resistive element, that allows passage of electrode energy through the layer 125. The layer 125 can be directly applied to the cutting element 123 and the electrode material 124. In some cases, an adhesive can be used to apply the layer 125 to the device 110.

As discussed herein, the non-stick coating can be applied to portions of the electrosurgical device to provide tissue adherence resistant (anti-stick) properties. Any material capable of providing the desired functionality (namely, reduction of tissue sticking while simultaneously maintaining sufficient electrical transmission to permit tissue sealing) may be used as the non-stick coating, provided it has adequate biocompatibility. In some examples, the material may be porous to allow for electrical transmission.

In some cases, the layer 125 can include a polymeric-based coating, such as a fluoropolymer type coating. In some cases, the layer 125 can include a Polytetrafluoroethylene (PTFE) coating. In some cases, the layer 125 can include a polysiloxane or a fluorosilane coating. For example, materials such as silicone and silicone resins can be used for the non-stick coating. In one example, the silicone and silicone resins can be applied using a plasma deposition process to precisely control thickness, and can withstand the heat generated during tissue sealing. Silicone resins suitable for the non-stick coating include, but are not limited to, polydimethyl siloxanes, polyester-modified methylphenyl polysiloxanes, such as polymethylsilane and polymethylsiloxane, and hydroxyl functional silicone resins. In some examples, the non-stick coating is made from a composition including a siloxane, which may include hexamethyldisiloxane, tetramethylsilane, hexamethyldisilazane, or combinations thereof.

In an example, the non-stick coating is a polydimethylsiloxane (“PMDSO” coating. In one example, the non-stick coating is a hexamethyldisiloxane (“HMDSO”) coating. In another example, the non-stick coating is a tetramethyldisiloxane (TMDSO or TMDS). In some cases, the layer 125 can include a thin layer of hexamethyldisiloxane (HMDSO), of a thickness of a few nano meters. HMDSO is electrically resistive, but the thinness of the coating can allow passage of RF energy therethrough.

The application of the non-stick coating may be accomplished using any system and process capable of precisely controlling the thickness of the coating. In some examples, HMDSO is deposited on the electrically conductive sealing plates using plasma enhanced chemical vapor deposition (PECVD) or other suitable methods such as atmospheric pressure plasma enhanced chemical vapor deposition (AP-PECVD). For example, the application of the polydimethylsiloxane coating may be accomplished using a system and process that includes a plasma device coupled to a power source, a source of liquid and/or gas ionizable media (e.g., oxygen), a pump, and a vacuum chamber. The power source may include any suitable components for delivering power or matching impedance to the plasma device. More particularly, the power source may be any radio frequency generator or other suitable power source capable of producing electrical power to ignite and sustain the ionizable media to generate a plasma effluent. Application of the coating is discussed in more detail below with reference to FIG. 9.

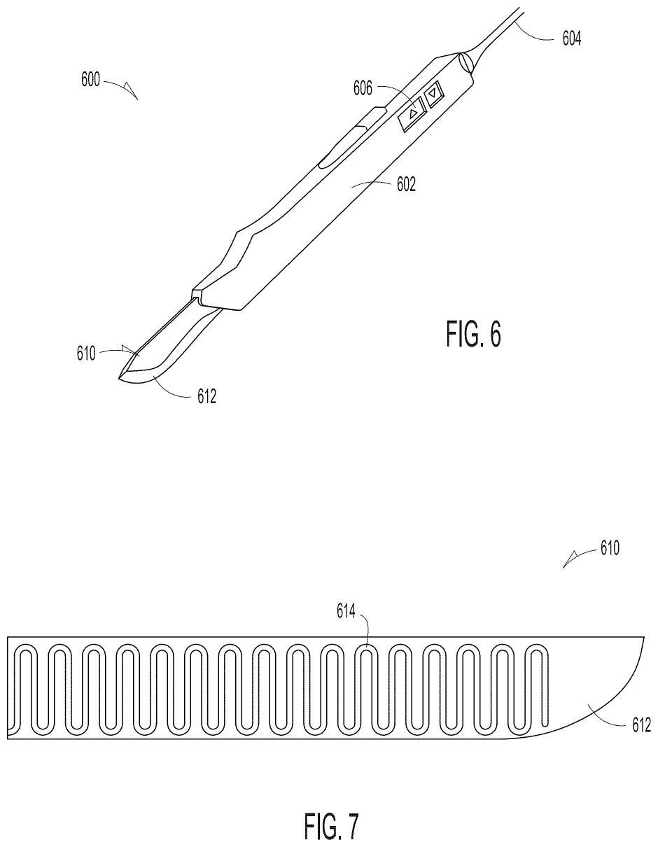

In some cases, the non-stick layer 125 can include an etched coating including one or more hydrophobic pillars superimposed on the electrode material 124. With an etched layer 125, a nanostructure of hydrophobic pillars can act as a superhydrophobic coating with a low surface energy, reducing sticking. The etched layer 125 can be in any suitable pattern for the non-stick coating to reduce or prevent tissue sticking. The etched layer 125 can be applied, for example, by printing, chemical etching, laser etching, chemical bombardment, or other suitable techniques. Application of the coating is discussed in more detail below with reference to FIGS. 6-8.

In some cases, it may be beneficial to have different hydrophobic physical structures on different surfaces of components of the device. The hydrophobic physical structure may be on all or a portion of a surface of the device 110, and different hydrophobic physical structures may be used on different surfaces or components of a device. Example hydrophobic structures are discussed below in FIGS. 6-8.

FIGS. 2-5 illustrate various electrosurgical devices containing cutting elements. Each of the cutting elements in these devices can have a non-stick layer to prevent build-up of tissue and preserve cutting quality.

FIG. 2 illustrates a distal portion 216 of a vessel sealing forceps 220 with a non-stick layer 225. The surgical jaw 220 can include jaws 222a, 222b, electrode plates 224a, 224b, cutting element 223, non-stick layer 225, flanges 226a, 226b, pivot point 227, and channel 228.

In vessel sealing forceps 220, the jaws 222a, 222b, can be hinged opposite each other and actuatable via one or more controls on the handpiece. The user can open and close the jaws 222a, 222b, as desired during surgery. The electrode plates 224a, 224b can be on either jaw 222a, 222b, to allow application of current from the generator to the target tissue, such as for vessel sealing. For example, during operation, the surgeon can close the jaws 222a, 222b, around the target tissue, and activate current flow to the electrode plates 224a, 224b, which can seal the tissue. The forceps 220 jaws 222a can be articulated, for example, through movement of the flanges 226a, 226b, around the pivot point 227.

The cutting element 223 can be configured to move in and out of a channel 228 in the body of the distal portion 216 of the device. When the forceps jaws 222a, 222b, have been used to seal tissue, the surgeon can activate extension of the cutting element 223 outward between the jaws 222a, 222b. The cutting element 223 can extend through the channel and cut the target tissue.

The non-stick layer 225 can be on and around at least a portion of the cutting element 223. In some cases, the non-stick layer 225 can be in and around the cutting channel 228 to prevent tissue build-up in the channel 228 when the cutting element 223 is moved in and out of the channel.

FIG. 3 illustrates a distal portion 316 of a dissecting forceps 320 with a cutting element 323 and a non-stick layer 325 in an example. The dissecting forceps 320 can include curved jaws 322a, 322b, electrode plates 324a, 324b, and non-stick layer 325. In dissecting forceps 320, the jaws themselves can act as the cutting element.

The dissecting forceps 320 can be similar to the forceps discussed above, but can be used for dissection of tissue. The jaws 322a, 322b, can be curved to allow for surgeon articulation and grasping of tissue. This can allow for secure grasping, dissecting, retracting, and coagulating of tissue. The plates 324a, 324b, can be serrated to allow for these actions. The plates 324a, 324b, can be fully or partially coated with the non-stick layer 325. In some cases, other types of forceps, such as a cutting forceps, can alternatively be used. In this case, the cutting portion of the forceps would be at least partially coated with the non-stick layer 325.

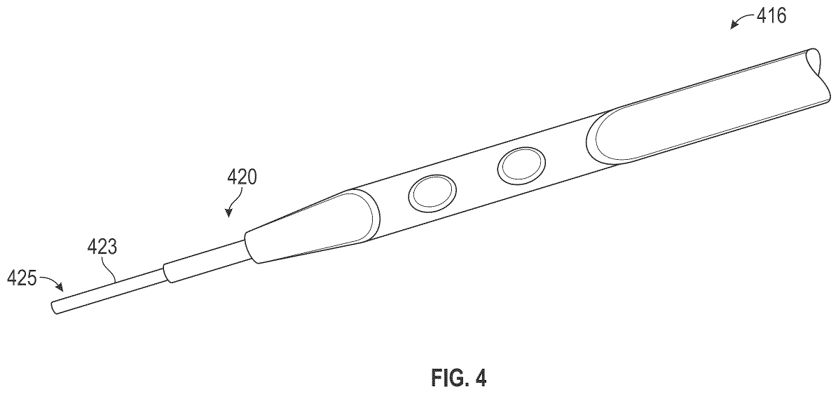

FIG. 4 illustrates a distal portion 416 of a cutting pencil 420 with a cutting element 423 and a non-stick layer 425 in an example. The pencil 420 itself can be the cutting element in this case, and can be at least partially coated by the non-stick layer 425. The pencil 420 can be connected to a generator, and be used for precise surgical techniques as desired. Alternatively, the non-stick layer can be used on the cutting portion of an electrosurgical spatula.

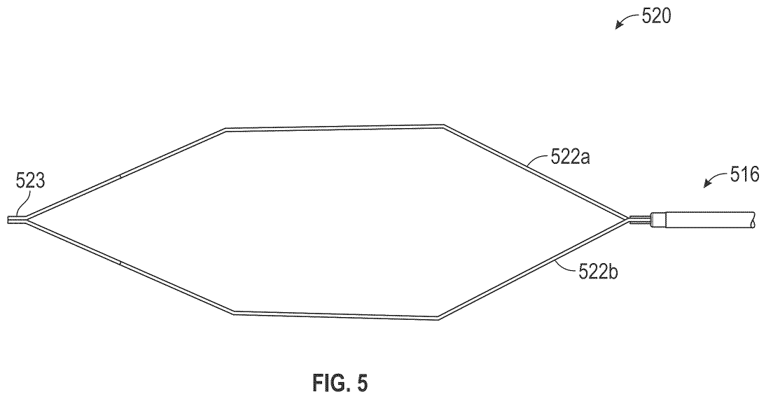

FIG. 5 illustrates a distal portion 516 of a laparoscopic loop with a mechanical cutting element 523 and a non-stick layer 525 in an example. The laparoscopic loop 516 can include loop section 522a, 522b, which meets at cutting element 523. The cutting element 523 can be at least partially covered by the non-stick layer 525. The laparoscopic loop 520 can be coupled to a generator and provide bipolar energy, such as for supracervical hysterectomy procedures.

FIG. 6 illustrates an example medical cutting device that includes a hydrophobic physical structure according to one example. As used in the following description, the term hydrophobic physical structure may refer to a collection of structures (for example, asperities) that collectively form the hydrophobic physical structure. FIG. 6 shows a medical cutting device 600 that includes a holder 602, and controls 606, for example, to control a heating element as discussed in more detail below. In the example of FIG. 6, the holder 602 is a scalpel handle, although the invention is not so limited. In other examples, the medical cutting device may be utilized in other more complex device configurations where the medical cutting device is not a scalpel.

The medical cutting device 600 includes a cutting assembly 610 coupled to the holder 602 at a distal end. The cutting assembly 610 includes a cutting blade 612 and a heating element 614 in thermal proximity to the cutting blade 612. In FIG. 6, a power cord 604 is further illustrated to supply energy to the heating element 614.

FIG. 7 shows a close-up view of the cutting assembly 610 from FIG. 6. The heating element 614 is shown in thermal proximity to the cutting blade 612. In one example, the heating element 614 is a resistive heating element. Other examples of heating elements include, but are not limited to, one or more RF electrodes that may induce heat in nearby tissue. In the example of FIG. 7, the heating element 614 is shown with a serpentine pattern, although the invention is not so limited. Other geometries of heating element 614 that provide sufficient heating are also possible. In one example, a serpentine provides a small cross-sectional area that heats up due to resistance at least in part from the reduced cross-sectional area. The length of the serpentine provides a larger surface area with which to transfer heat from the heating element 614 to adjacent tissue and/or the blade 612.

In one example, the heating element 614 forms a direct interface with the blade 612. In one example, the heating element 614 is separated from the blade 612 by one or more dielectric structures such as an isolation layer. In the present disclosure, terms such as “dielectric,” and “conductor” are relative. For example, a conductor may be defined as a material that is more conductive than another material that is less conductive. Likewise, a dielectric, or insulator may be defined as a material that is less conductive than another material that is more conductive. One example of an isolation layer may include a polymer layer such as polytetrafluoroethylene (PTFE), silicone, or other electrically isolating layer. In one example, an isolation layer may include a ceramic material, such as an oxide layer. In this description, thermal proximity may include a direct interface between the heating element 614 and the blade 612. Thermal proximity may also include a close proximity between the heating element 614 and the blade 612. Either configuration allows tissue near the blade to be heated to induce cauterization and/or coagulation.

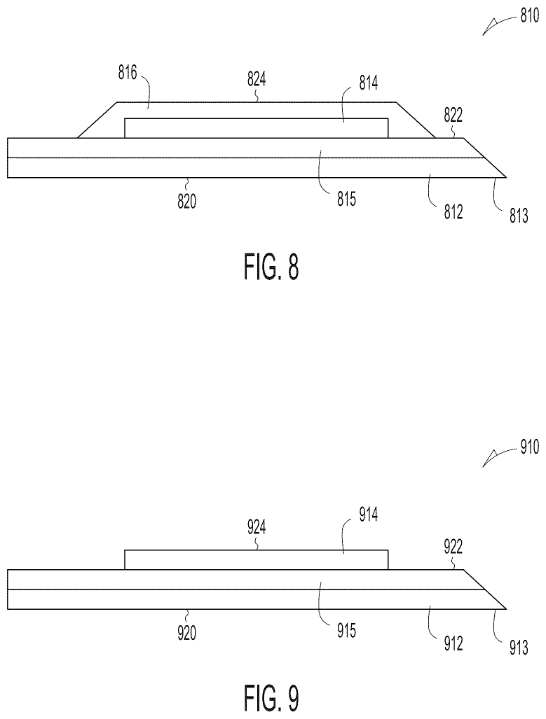

FIG. 8 shows a cross section of a cutting assembly 810, similar to cutting assembly 610 shown in FIG. 6. In one example, the cutting assembly 810 may be described as a plurality of laminate layers. Although selected layers are shown, other examples may include additional layers, or fewer layers than are illustrated in FIG. 8. The cutting assembly 810 includes a cutting blade 812, having a sharpened edge 813. In one example, the cutting assembly 810 is formed from stainless steel. Other example materials may include, but are not limited to, titanium, carbon steel, ceramic materials, etc. In the example, of FIG. 8, an isolation layer 815 is included and forms a direct interface with the cutting blade 812. Examples of isolation layer 815 include, but are not limited to, PTFE, silicone, other polymers, ceramic, or other thermally insulating materials.

A heating element 814 is shown in thermal proximity to the cutting blade 812. In the example shown, the heating element 814 is physically separated from the cutting blade 812 by the isolation layer 815 but is close enough to an incision when in use to provide thermal heating to either the incision, the cutting blade 812 or both. In one example, the heating element 814 illustrated in FIG. 8 is part of a serpentine resistive heating element similar to the heating element 614 shown in FIGS. 1 and 2.

In the example of FIG. 8, an outer coating 816 covers all or a portion of at least one side of the cutting assembly 810. As shown in FIG. 8, the outer coating 816 covers the heating element 814 completely and covers a portion of the isolation layer 815 on either side of the heating element 814. In one example, the outer coating 816 is a polymer. In one example, the outer coating 816 is a monomer. In one example, the outer coating 816 is a ceramic or other dielectric. One example of a polymer outer coating includes PTFE, although the invention is not so limited.

In one example, a surface 824 of the outer coating 816 includes a hydrophobic physical structure. In one example, the outer coating 816 is modified to include a hydrophobic physical structure as described in more detail below. In one example, modifying to include a hydrophobic physical structure includes depositing a separate layer with a hydrophobic physical structure over the outer coating 816. In one example, modifying to include a hydrophobic physical structure includes etching or otherwise altering a surface from the bulk of the outer coating 816 to form the hydrophobic physical structure.

In one example, other surfaces of other components in the cutting assembly 810 are modified to include a hydrophobic physical structure. For example, surface 820 of the cutting blade 812 may be modified to include a hydrophobic physical structure. Likewise, surface 822 of isolation layer 815 may be modified to include a hydrophobic physical structure. All surfaces 820, 822, and 824 may be modified to include a hydrophobic physical structure, or only selected surfaces 820, 822, and 824 may be modified to include a hydrophobic physical structure. Additionally, all, or only part of each surface 820, 822, and 824 may be modified to include a hydrophobic physical structure.

FIG. 9 shows another cross section of a cutting assembly 910. In one example, the cutting assembly 910 may be described as a plurality of laminate layers. Although selected layers are shown, other examples may include additional layers, or fewer layers than are illustrated in FIG. 9. The cutting assembly 910 includes a cutting blade 912, having a sharpened edge 913. Similar to FIG. 8, in the example of FIG. 9, an isolation layer 915 is included and forms a direct interface with the cutting blade 912. A heating element 914 is shown in thermal proximity to the cutting blade 912. In one example, the heating element 914 illustrated in FIG. 9 is part of a serpentine resistive heating element similar to the heating element 614 shown in FIGS. 1 and 2.

In one example, a surface 924 of the heating element 914 includes a hydrophobic physical structure. In one example, modifying to include a hydrophobic physical structure includes etching or otherwise altering a surface from the bulk of the heating element 914 to form the hydrophobic physical structure. Etching or otherwise altering a surface from the bulk is illustrated in more detail in discussion of FIG. 5 below.

Similar to FIG. 8, in one example, other surfaces of other components in the cutting assembly 910 are modified to include a hydrophobic physical structure. For example, surface 920 of the cutting blade 912 may be modified to include a hydrophobic physical structure. Likewise, surface 922 of isolation layer 915 may be modified to include a hydrophobic physical structure. All surfaces 920, 922, and 924 may be modified to include a hydrophobic physical structure, or only selected surfaces 920, 922, and 924 may be modified to include a hydrophobic physical structure. Additionally, all, or only part of each surface 920, 922, and 924 may be modified to include a hydrophobic physical structure.

In one example, the term hydrophobic physical structure is in contrast to a chemical coating, lubricant, or other hydrophobic layer whose principal of operation is based on chemistry. In one example, hydrophobic physical structures include nanoscale structures that provide hydrophobicity as described in more detail below.

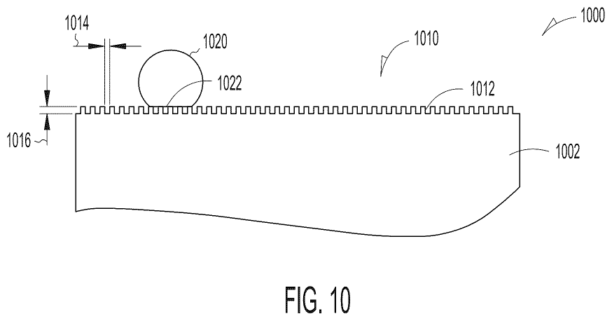

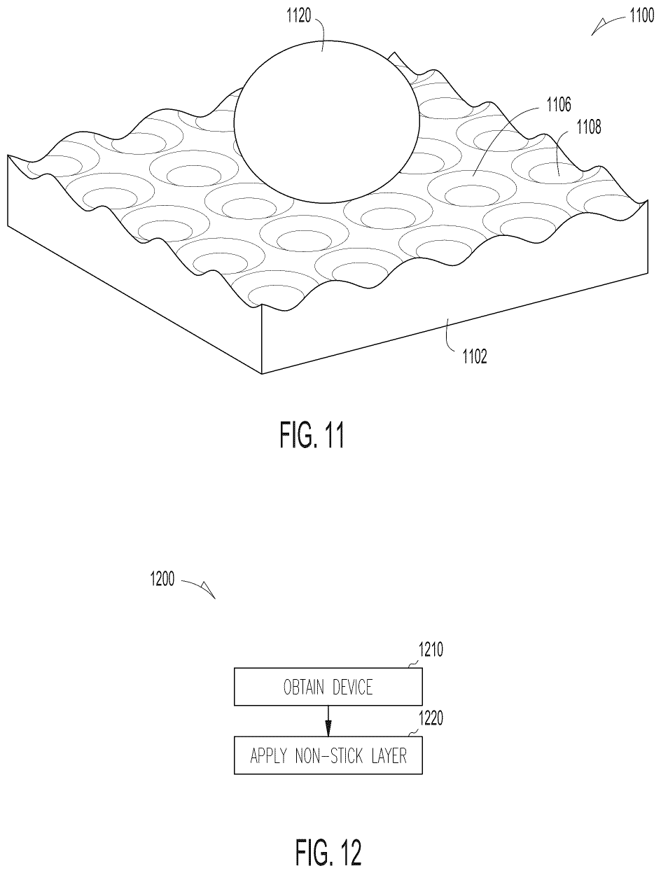

FIGS. 10-11 depict schematic diagrams of various examples of a non-stick layer that can be used on insulation elements of electrosurgery cutting device end effectors.

As shown in FIG. 10 in one example, the hydrophobic physical structure 1010 includes asperities 1012 having a height 1016 and a pitch 1014. The hydrophobic physical structure 1010 can be described by the following equation:

$${Λ}_{c} = \frac{- ρ {{gV}^{1 / 3} ( ( \frac{1 - cos ( {θ}_{a} )}{sin ( {θ}_{a} )} ) ( 3 + {( \frac{1 - cos ( {θ}_{a} )}{sin ( {θ}_{a} )} )}^{2} ) )}^{2 / 3}}{{( 36 π )}^{1 / 3} γ cos ( {θ}_{a , 0} + w - 90 ) )}$$

where Λ is a contact line density, and Λ_{c} is a critical contact line density; ρ=density of the liquid droplet; g=acceleration due to gravity; V=volume of the liquid droplet; θₐ=advancing apparent contact angle; θ_{a,0}=advancing contact angle of a smooth substrate; γ=surface tension of the liquid; and w=tower wall angle.

The contact line density A is defined as a total perimeter of asperities over a given unit area.

In one example, if Λ>Λ_{c} then a droplet 1020 of liquid are suspended in a Cassie-Baxter state. Otherwise, the droplet 1020 will collapse into a Wenzel state. In one example when a Cassie-Baxter state is formed, an ultra-hydrophobic condition exists, and a low adhesion surface is formed. FIG. 10 illustrates a Cassie-Baxter state, where the droplet 1020 rests on top of the asperities 1012 at interface 1022. Although rectangular asperities are shown for illustration purposes, the invention is not so limited. Asperity shapes are taken into account in the formula above, at least in the tower wall angle (w) term.

In the example of FIG. 10, the asperities are formed directly from a bulk material, and are not formed from a separate coating. One method of forming asperities directly from a bulk material includes chemical etching. Another example of forming asperities directly from a bulk material includes laser etching or ablation. Another example of forming asperities directly from a bulk material includes ion etching.

FIG. 11 shows one example of a laser etched surface 1100 that includes hydrophobic physical structure as described above. In the example of FIG. 11, a gaussian hole array is formed by applying laser energy to a surface of a substrate 1102 in a controlled regular pattern to form holes 1106. A shape of the holes 1106 is characterized as gaussian due to the energy distribution of laser energy in forming the array. In the example shown, a number of asperities 1108 are formed in the process that may be spaced and arranged in an array that provides a Cassie-Baxter state as described above. A liquid droplet 1120 is illustrated on the hydrophobic physical structure like the droplet from FIG. 11.

FIG. 12 is a flow chart depicting a method 1200 of applying a hydrophobic coating to a surgical device. The method 1200 can include obtaining an electrosurgery cutting device (block 1210) and applying a non-stick layer (1220).

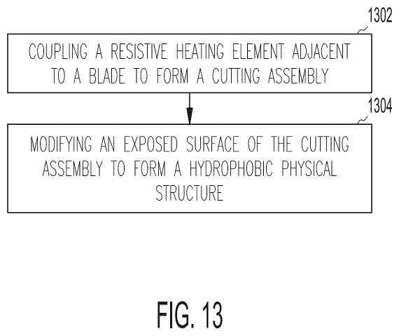

FIG. 13 shows a flow diagram of an example method of forming a medical device including a hydrophobic physical structure. In operation 1302 a resistive heating element is coupled adjacent to a blade to form a cutting assembly. In operation 1304 an exposed surface of the cutting assembly is modified to form a hydrophobic physical structure.

The methods 1200 and 1300 can include coating or etching the surface of cutting elements of a bipolar cutting device with a non-stick layer, such that the layer at least partially covers the cutting element. Application of the coating or layer can be done, for example, by chemical etching, laser etching, chemical bombardment, or printing.

In some cases, the coating can be produced in a uniform thickness of about 1 nm to about 300 nm, of about 5 nm to about 200 nm, or of about 10 nm to about 100 nm. In some cases, the coating can be produced in a pattern, such as to create hydrophobic pillars on the electrode. In some cases, the coating can fully or partially cover the electrode.

Several modification/application techniques may be used to form the coating, optionally including hydrophobic pillars. In one example, a sol-gel process can be used. Advantages of sol-gel application include the ability to coat more complex surfaces with high quality films. Challenges of sol-gel may include brittleness, limited thickness options, and induced mechanical stresses in the coating.

In one example, a cold spray process can be used. Advantages of cold spray application include the ability to coat at lower temperatures, with low deterioration, low oxidation, and low defects. Challenges of cold spray may include high energy needed for application, high cost, and a limited number of compatible substrates.

In one example, a chemical vapor deposition (CVD) process can be used. Advantages of CVD application include a high-quality coating, high control of thickness, and the ability to coat complex surfaces. Challenges of CVD may include high temperature requirements, and high cost.

In one example, a physical vapor deposition (PVD) process can be used. Advantages of PVD application include the ability to coat inorganic compounds, ecological friendly processes, and a wide variety of available coating materials. Challenges of PVD may include high vacuum chamber requirements and high cost.

In one example, a thermal spray process can be used. Advantages of thermal spray application include a large selection of compatible coating materials and substrate materials, and low cost. Challenges of thermal spray may include difficulty in forming thick coatings, low adhesion issues of coatings, and ecologically unfriendly process steps.

In one example, an in-situ polymerization process can be used. Advantages of in-situ polymerization include the ability to coat with insoluble polymers. Challenges of in-situ polymerization may include process complexity, high cost, and limited potential for large scale production.

In one example, a spin coating process can be used. Advantages of spin coating include high quality coatings, fast drying times, and controllable thicknesses. Challenges of spin coating may include difficulty coating small surfaces and requirements of a smooth surface.

In one example, a dip coating process can be used. Advantages of dip coating include the ability to coat complex surfaces and the ability for large scale production. Challenges of dip coating may include undesirable solvent requirements, and limitations of only soluble polymer coatings.

In one example, an electrodeposition process can be used. Advantages of electrodeposition include high quality coatings at low cost. Challenges of electrodeposition may include long process times, and conductive substrate requirements.

Medical devices having a non-stick coating optionally including hydrophobic pillars as described show reduced adhesion over other non-textured coatings for biomaterials including, but not limited to, tissues, blood, fats, and/or other biological materials. Application of hydrophobic physical structures to other surfaces of medical devices apart from optical components may further provide advantages such as reduced friction and reduced adhesion where desired.

### VARIOUS NOTES & EXAMPLES

Example 1 can include an electrosurgical device, comprising: a longitudinal shaft having a distal portion and a proximal portion, the shaft for at least partial insertion into a patient; an end effector on the distal portion of the longitudinal shaft, wherein the end effector is configured to cut tissue, the end effector having an electrically conductive component configured to operably couple to a source of electrosurgical energy for treating tissue, and a mechanical cutting element; and a non-stick layer at least partially covering the cutting element, wherein the non-stick layer comprises a material having a surface adherence to tissue that is less than a surface adherence to tissue of the material of the cutting element.
Example 2 can include Example 1, wherein the electrosurgical device comprises a vessel sealing forceps, the end effector comprising a first jaw member and an opposing second jaw member, wherein the cutting element extends therebetween.
Example 3 can include any of Examples 1-2, further comprising a channel extending down the longitudinal shaft, the cutting element configurable for movement in and out of the channel, wherein the non-stick layer at least partially covers the channel.
Example 4 can include any of Examples 1-3, wherein the electrosurgical device comprises a dissecting forceps, the end effector comprising a curved first jaw member and an opposing curved second jaw member, wherein the cutting component extends therebetween.
Example 5 can include any of Examples 1-4, wherein the electrosurgical device comprises a surgical pencil, the end effector comprising a pencil extending from the longitudinal shaft, wherein the cutting element comprise the pencil.
Example 6 can include any of Examples 1-5, wherein the electrosurgical device comprises a laparoscopic loop, the end effector comprising a looped component, wherein the cutting element comprises the looped element.
Example 7 can include any of Examples 1-6, wherein the electrically conductive component comprises one or more electrodes configured to apply energy to target tissue.
Example 8 can include any of Examples 1-7, wherein the mechanical cutting element is separate from the one or more electrodes.
Example 9 can include any of Examples 1-8, wherein the non-stick layer has a thickness within a range of about 10 nm to about 30 nm.
Example 10 can include any of Examples 1-9, wherein the non-stick layer has a substantially uniform thickness.
Example 11 can include any of Examples 1-10, wherein the non-stick layer is continuous.
Example 12 can include any of Examples 1-11 wherein the non-stick layer, wherein the non-stick layer is selected from one of polydimethylsiloxane, hexadimethylsiloxane, and tetramethyldisiloxane.
Example 13 can include any of Examples 1-12 wherein the non-stick layer comprises a non-conductive material.
Example 14 can include any of Examples 1-13, wherein the non-stick layer comprises an electrically insulating material.
Example 15 can include any of Examples 1-14, wherein the non-stick layer comprises a hydrophobic surface structure, the hydrophobic surface structure having a lower surface adherence than the insulation element.
Example 16 can include any of Examples 1-15, wherein the non-stick layer comprises a hydrophobic surface structure and a coating.
Example 17 can include any of Examples 1-16, wherein the non-stick layer overlays a portion of the electrically conductive component.
Example 18 can include a method comprising applying a non-stick layer to a mechanical cutting element of an end effector in a surgical device, wherein the non-stick layer comprises a hydrophobic surface.
Example 19 can include Example 18, wherein applying a non-stick layer comprises etching a hydrophobic surface structure.
Example 20 can include any of Examples 18-19, wherein depositing a non-stick layer comprises applying a coating to the mechanical cutting element.
Example 21 includes a medical cutting device. The medical cutting device includes a holder, and a cutting assembly coupled to the holder. The cutting assembly includes a cutting blade and a heating element in thermal proximity to the cutting blade, and a hydrophobic physical structure on at least a portion of the cutting assembly.
Example 22 includes the medical cutting device of example 1, wherein the heating element is electrically isolated from the blade.
Example 23 includes the medical cutting device of any one of examples 1-2, wherein the hydrophobic physical structure is part of an external layer in a plurality of laminate layers.
Example 24 includes the medical cutting device of any one of examples 1-3, wherein the hydrophobic physical structure is part of an external coating that is layered over an insulator coating.
Example 25 includes the medical cutting device of any one of examples 1-4, wherein the holder includes a scalpel handle.
Example 26 includes the medical cutting device of any one of examples 1-5, wherein the hydrophobic physical structure region is part of a bulk material that forms the cutting blade.
Example 27 includes the medical cutting device of any one of examples 1-6, wherein the hydrophobic physical structure is part of a bulk material that forms the heating element.
Example 28 includes the medical cutting device of any one of examples 1-7, wherein the hydrophobic physical structure includes a gaussian hole array.
Example 29 includes the medical cutting device of any one of examples 1-8, wherein the hydrophobic physical structure is part of a coating that covers at least a portion of the cutting assembly.
Example 30 includes the medical cutting device of any one of examples 1-9, wherein the coating includes a non-uniform thickness.
Example 31 includes the medical cutting device of any one of examples 1-10, wherein the coating is electrically conductive.
Example 32 includes the medical cutting device of any one of examples 1-11, wherein the coating is a dielectric.
Example 33 includes the medical cutting device of any one of examples 1-12, wherein the coating includes polysiloxane.
Example 34 includes the medical cutting device of any one of examples 1-13, wherein the coating includes hexamethyldisiloxane (HMDSO).
Example 35 includes the medical cutting device of any one of examples 1-13, wherein the coating includes fluorosilane.
Example 36 includes a method of forming a resistive heating cutting blade. The method includes coupling a resistive heating element adjacent to a blade to form a cutting assembly and modifying an exposed surface of the cutting assembly to form a hydrophobic physical structure.
Example 37 includes the method of example 16, wherein modifying the exposed surface of the cutting assembly includes etching a portion of the blade.
Example 38 includes the method of any one of example 16-17, wherein modifying the exposed surface of the cutting assembly includes etching a portion of the resistive heating element.
Example 39 includes the method of any one of example 16-18, wherein modifying the exposed surface of the cutting assembly includes depositing a coating.
Example 40 includes the method of any one of example 16-19, wherein depositing a coating includes chemical vapor deposition (CVD).
Example 41 includes the method of any one of example 16-20, wherein depositing a coating includes physical vapor deposition (PVD).
Example 42 includes the method of any one of example 16-21, wherein modifying the exposed surface of the cutting assembly further includes modifying a surface of the coating after deposition.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to conFIG. an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A medical cutting device comprising:
a longitudinal shaft having a distal portion and a proximal portion, the distal portion including:
a cutting channel extending distally from the proximal portion of the longitudinal shaft and defining a lumen therethrough;
a surgical jaw coupled to the distal portion of the shaft, the surgical jaw comprising:
a cutting blade configurable for movement in and out of the channel;
one or more electrode plates, wherein the one or more electrode plates are electrically isolated from the cutting blade; and
an arrangement of homogenous hydrophobic physical structures defining adjacent like-height peaks interposed by corresponding like-depth valleys such that the adjacent peaks are separated by a uniform pitch at least partially covering the cutting blade and an interior surface of the lumen defined by the cutting channel.

2. The medical cutting device of claim 1, wherein the hydrophobic physical structures are part of an external layer in a plurality of laminate layers.

3. The medical cutting device of claim 1, wherein the hydrophobic physical structures are part of an external coating that is layered over an insulator coating on a heating element.

4. The medical cutting device of claim 1, wherein the hydrophobic physical structures are part of a bulk material that forms the cutting blade.

5. The medical cutting device of claim 3, wherein the hydrophobic physical structures are part of a bulk material that forms the heating element.

6. The medical cutting device of claim 1, wherein the hydrophobic physical structures are part of a coating that covers at least a portion of the cutting assembly.

7. The medical cutting device of claim 1, wherein the heating element is separated from the cutting blade by an isolation layer comprising one or more dielectric structures.

8. The medical cutting device of claim 7, further comprising an arrangement of homogenous hydrophobic physical structures on the cutting blade, on the cutting assembly, and on the isolation layer.

9. The medical cutting device of claim 7, wherein the arrangement of homogenous hydrophobic physical structures has a lower surface adherence than the isolation layer.

10. The medical cutting device of claim 1, wherein individual ones of the hydrophobic physical structures each have an equal height and an equal pitch.

11. The medical cutting device of claim 1, wherein the arrangement of homogenous hydrophobic physical structures is configured as a Gaussian hole array.
